(19) United States
(12) Patent Application Publication
(10) Pub. No.: US 2009/0149655 A1
(43) Pub. Date: Jun. 11, 2009

(54) Process for the preparation of Retapamulin and its intermediates

(76) Inventors: Lilach Hedvati, Doar Na Hefer (IL); Eyal Gilboa, Bat-Yam (IL); Sharon Avhar-Maydan, Givataym (IL); Sharona Shachan-Tov, Kfar-Saba (IL)

Correspondence Address: KENYON & KENYON LLP, ONE BROADWAY, NEW YORK, NY, 10004, US

(21) Appl. No.: 12/315,782

(22) Filed: Dec. 5, 2008

Related U.S. Application Data
(60) Provisional application No. 61/005,638, filed on Dec. 5, 2007.
(60) Provisional application No. 61/132,430, filed on Jun. 17, 2008.

(51) Int. Cl. C07D 451/06

(52) U.S. Cl. 546/128; 546/130

(57) ABSTRACT

Processes for preparing tropine derivatives of the following Formula B

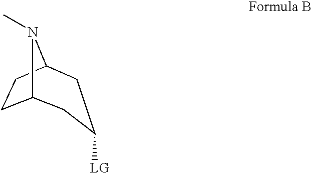

are provided where LG represents a leaving group. The tropine derivatives may be used to prepare Retapamulin, preferably in substantially pure form. Also provided are processes for preparing Retapamulin comprising combining a base, tropine thiol in a free base or salt form, an organic solvent, and a pleuromutilin derivative of Formula A:

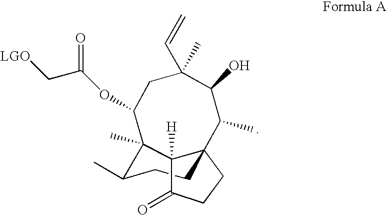

### CROSS REFERENCE TO RELATED APPLICATIONS

This patent application claims the benefits of U.S. Provisional Application No. 61/005,638 filed on Dec. 5, 2007 and 61/132,430 filed on Jun. 17, 2008, the disclosures of which are hereby incorporated by reference.

### FIELD OF THE INVENTION

The invention encompasses processes for preparing Retapamulin and its intermediates.

### BACKGROUND OF THE INVENTION

Retapamulin [CAS number: 224452-66-8] has the chemical name 5-Acetic acid, [[(3-exo)-8-methyl-8-azabicyclo[3.2.1]oct-3-yl]thio]-,(3aS,4R,5S,6S,8R,9R,9aR,10R)-6-ethenyldecahydro-5-hydroxy-4,6,9,10-tetramethyl-1-oxo-3a,9-propano-3aH-cyclopentacycloocten-8-yl ester and the following chemical structure:

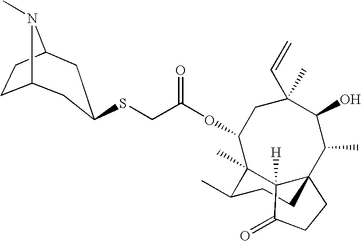

Retapamulin was first disclosed in U.S. Pat. No. 6,281,226 and is used in the treatment of secondarily-infected traumatic lesions (“SITL”). Processes for the preparation of Retapamulin have been disclosed in International Patent Application Publication No. WO 99/21855 and U.S. Patent Application Publication No. 2006/0276503. There is a need in the art for improved processes for preparing Retapamulin and its intermediates, which are more industrially applicable.

### SUMMARY OF THE INVENTION

In one embodiment, the present invention encompasses a process for preparing a tropine derivative of the following Formula B:

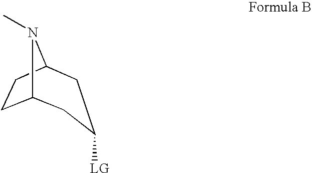

comprising replacing the hydroxyl group of tropine with a leaving group in the presence of an organic solvent selected from the group consisting of C₂-C₆ ketones, C₂-C₇ esters and C₄-C₈ ethers.

In another embodiment, the invention encompasses a process for preparing Retapamulin comprising: combining a base, tropine thiol in a free base or salt form, an organic solvent and a pleuromutilin derivative of Formula A:

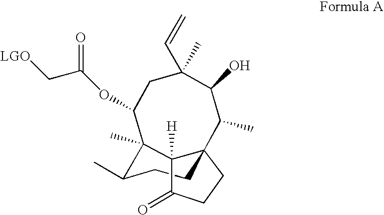

Preferably, the Retapamulin obtained is substantially pure.

In another embodiment, the invention encompasses a process for preparing Retapamulin comprising: replacing the hydroxyl group in the β position relative to the carbonyl group of pleuromutilin with a leaving group (“LG”) in the presence of an organic solvent such as C₆-C₉ aromatic hydrocarbons, C₂-C₆ ketones, C₂-C₇ esters and C₄-C₈ ethers to obtain a pleuromutilin derivative of Formula A; admixing the pleuromutilin derivative with a base, tropine thiol in free or salt form, and an organic solvent to obtain Retapamulin. Preferably, the Retapamulin obtained is substantially pure.

### DETAILED DESCRIPTION OF THE INVENTION

As used herein, the term “substantially pure Retapamulin” refers to Retapamulin having total chemical purity of above 90% as measured by HPLC. Preferably, the substantially pure Retapamulin has a total purity of above 95%, most preferably, above 99%.

As used herein the term “volume” refers to ml of solvent per gram of material.

As used herein, the term “room temperature” refers to a temperature of about 20° C. to about 35° C., more preferably about 20° C. to about 25° C., and most preferably about 25° C.

In one embodiment, the present invention encompasses a process for preparing a tropine derivative of the following Formula B:

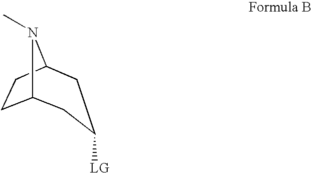

comprising: replacing the hydroxyl group of tropine with a leaving group (LG) in the presence of an organic solvent selected from the group consisting of C₂-C₆ ketones, C₂-C₇ esters and C₄-C₈ ethers. Preferably, the tropine derivative is tropine mesylate.

The hydroxyl group of tropine is typically in the axial position.

Any reagent capable of replacing the hydroxyl group with a leaving group may be used in the above process. Examples of suitable reagents include, but are not limited to, methane sulphonyl chloride, p-toluenesulfonyl chloride (“TsCl”), thionyl chloride (SOCl₂), thionyl bromide (“SOBr₂”), 4-nitrobenzenesulfonyl chloride (“nosyl chloride;” “NsCl”), benzenesulfonyl chloride, acetyl chloride (“AcCl”) and acetic anhydride (“Ac₂O”). Most preferably, the reagent is methane sulfonyl chloride.

Preferably, replacing the hydroxyl group is carried out in the presence of an organic base such as triethyl amine and tributyl amine. Most preferably, the base is triethyl amine. The organic solvent used is preferably selected from a group consisting of acetone, methyl isobutyl ketone, ethyl acetate, isobutyl acetate and tetrahydrofuran (THF). Most preferably, the organic solvent is THF or methyl isobutyl ketone.

The reaction mixture is typically maintained at a temperature of about −10° C. to about reflux.

In some embodiments, the tropine, the organic solvent, and the base may be combined at about room temperature. The reagent capable of replacing the hydroxyl group with a leaving group may then be added after, or while, the reaction is cooled to about −10° C. to about 5° C., preferably to about −3° C. or to about −5° C. The reaction may then be warmed to room temperature and maintained for a time, e.g., about 5-24 hours, about 8-16 hours, about 12-15 hours, or overnight.

The above obtained tropine derivative may be further converted to Retapamulin by any methods known in the art.

In a further embodiment, the present invention encompasses a process for preparing Retapamulin comprising obtaining a tropine derivative as described above and further converting it to Retapamulin. The conversion of the tropine derivative to Retapamulin may be performed by any method known in the art, such as combining the tropine derivative with an S-donor to obtain tropine thiol and further condensing tropine thiol with a pleuromutilin derivative to obtain Retapamulin.

In another embodiment, the invention encompasses a process for preparing Retapamulin. Typically, the process comprises: combining a base, tropine thiol in free base or salt form, an organic solvent and a pleuromutilin derivative of the following Formula A:

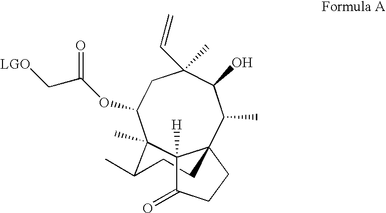

Preferably, the Retapamulin obtained is substantially pure.

The process described above for obtaining Retapamulin is performed in an organic solvent system, (for example, not a biphasic system) and does not involve the use of a phase transfer catalyst.

The organic solvent used in the reaction can be selected from the group consisting of: butanol, isopropanol, methyl tert butyl ether, acetone, acetonitrile, ethylacetate, toluene, and methyl isobutyl ketone

Bases, such as KOH and t-BuONa, are ionized and typically produce a pH range of about 7 to about 12. Typically, the base is an alkali base or carbonate such as t-BuONa, KOH, K₂CO₃, Na₂CO₃, or a trialkyl amine, such as Et₃N.

Preferably, the combination is maintained for about 2 to about 24 hours. Typically, the combination is maintained at a temperature sufficient to obtain the substantially pure Retapamulin, and preferably at a temperature of about 10° C. to about reflux temperature.

In another embodiment, the invention encompasses a process for preparing Retapamulin comprising: replacing the hydroxyl group in the β position relative to the carbonyl group of pleuromutilin with a leaving group (“LG”) in the presence of an organic solvent such as C₆-C₉ aromatic hydrocarbon, C₂-C₆ ketones, C₂-C₇ esters and C₄-C₈ ethers to obtain a pleuromutilin derivative of Formula A; admixing the pleuromutilin derivative with a base and tropine thiol in free base or salt form in an organic solvent to obtain Retapamulin. Preferably, the Retapamulin obtained is substantially pure.

The process above for obtaining Retapamulin can be carried out with or without the isolation of the pleuromutilin derivative.

Pleuromutilin starting material can be purchased or obtained by methods well-known in the art, including obtaining pleuromutilin as a fermentation product.

Any reagent capable of replacing the hydroxyl group in the β position relative to the carbonyl group of the pleuromutilin with a leaving group may be used in the above process. Examples of suitable reagents include, but are not limited to, methane sulphonyl chloride, p-toluenesulfonyl chloride (“TsCl”), thionyl chloride (SOCl₂), thionyl bromide (“SOBr₂”), 4-nitrobenzenesulfonyl chloride (“nosyl chloride”; “NsCl”), benzenesulfonyl chloride, acetyl chloride (“AcCl”) and acetic anhydride (“Ac₂O”).

Preferably, replacing of the hydroxyl group is carried out in the presence of an organic base such as secondary or tertiary amine. The secondary or tertiary amine can be selected from the group consisting of triethyl amine, di-isopropyl amine, and tributyl amine. Most preferably, the amine is triethyl amine.

Preferably, the organic solvent used in the process for obtaining the pleuromutilin derivative is selected from the group consisting of: toluene, ethyl acetate, isobutyl acetate, methyl isobutyl ketone, acetone and tetrahydrofuran (THF). Most preferably, the solvent is acetone.

Typically, the combination of the pleuromutilin, organic base, organic solvent and the reagent capable of replacing the hydroxyl group is maintained for a period of time and at a temperature sufficient to obtain the pleuromutilin derivative. Preferably, the combination is maintained for about 0.5 to about 24 hours, about 1 to about 16 hours, about 3 to about 12 hours, or about 5 to about 10 hours. Preferably, the combination is maintained at a temperature of about 0° C. to about reflux, about 5° C. to about reflux, or about 20° C. to about reflux.

Preferably, the pleuromutilin derivative is pleuromutilin mesylate.

The pleuromutilin derivative thus obtained may optionally be further purified by crystallization from isopropanol.

In one specific embodiment, the process for obtaining Retapamulin comprises: providing a mixture of pleuromutilin, an organic solvent such as toluene, ethyl acetate, isobutyl acetate, methyl isobutyl acetate acetone, methyl isobutyl ketone and tetrahydrofuran (THF), a secondary or tertiary amine selected from the group consisting of triethyl amine, di-isopropyl amine and tributyl amine, and methanesulfonyl chloride; maintaining the combination to obtain pleuromutilin mesylate; admixing the pleuromutilin mesylate with a base and tropine thiol in free or salt form in a solvent selected from the group consisting of butanol, isopropanol, methyl tert butyl ether, acetone, acetonitrile, ethylacetate, toluene, and methyl isobutyl ketone; and maintaining the mixture for a period of time sufficient to obtain Retapamulin.

One specific embodiment of the invention discloses a process for preparing Retapamulin which can be described by the following Scheme:

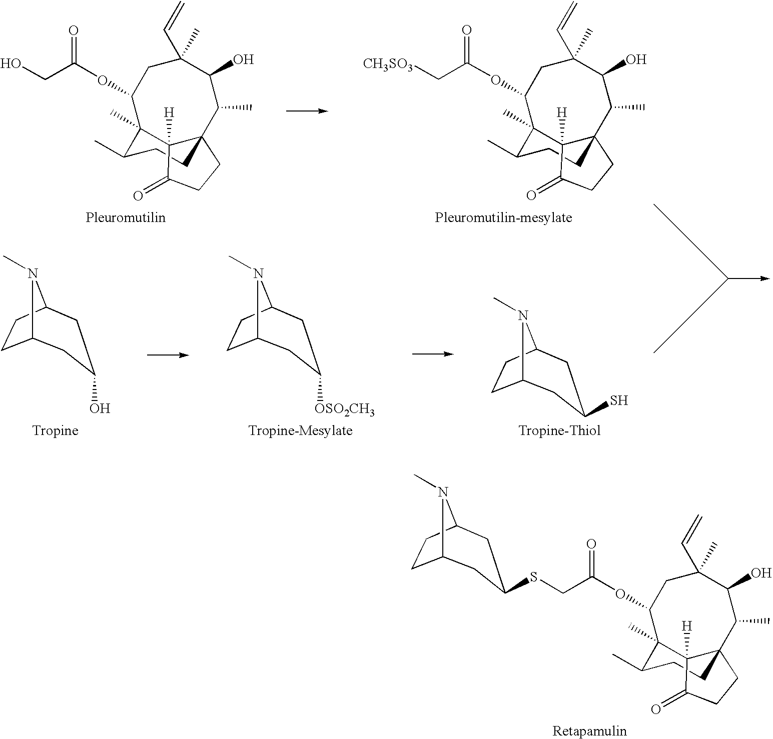

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Example 1

### Preparation of Pleuromutilin Mesylate

A flask (2 L) was loaded with pleuromutilin (120 g), methyl isobutyl ketone (MIBK) (800 ml) and Et₃N (60 ml). The solution was cooled to −3° C. and a solution of methanesulfonyl chloride (51 ml) in MIBK (150 ml) was added dropwise to the solution while maintaining the temperature between −3° C. and 10° C. The addition of the methanesulfonyl chloride resulted in the formation of a slurry. The slurry was heated slowly to 25° C. The slurry was then extracted with water (500 ml) and the phases were separated. The organic phase was washed with water (250 ml), washed with brine (130 ml) and dried with Na₂SO₄. The organic phase was then evaporated to dryness. The resulting residue was dissolved in isopropyl alcohol (IPA) (400 ml), the solution was heated to reflux and then cooled to room temperature to precipitate pleuromutilin mesylate. The precipitated pleuromutilin mesylate was filtered from the solution and dried at 50° C. in a vacuum oven overnight. Approximately 116 g of pleuromutilin mesylate were obtained.

### Example 2

### Preparation of Pleuromutilin Mesylate

A 3 neck-flask (250 ml) is charged with Pleuromutilin (PLM) (10 g), Toluene (70 ml) and triethyl amine (TEA) (4.6 ml). The mixture is cooled to 5° C. and a solution of methanesulfonyl chloride (2.5 ml) in toluene (20 ml) is added dropwise. The reaction mixture is stirred at room temperature overnight. Pleuromutilin mesylate is obtained in more then 95% conversion. The solution is used with no other purification.

### Example 3

### Preparation of Pleuromutilin Mesylate

A 250 ml round bottom flask was charged with Pleuromutilin (10 g, 0.0263 mol), Et₃N (4.2 ml, 0.02895 mol) and methyl isobutyl ketone (MIBK) (3 vol, 30 ml). Methane sulfonyl chloride (2 eq, 4 ml, 0.0515 mol) was added at room temperature. The solution became a massive white slurry. The reaction mixture was stirred for 2 hours at room temperature, and then filtered and washed with MIBK (10 ml).

n-Hexane (15 vol) was added and the mixture was stirred at room temperature overnight. The obtained precipitate was filtered under reduced pressure and dried under vacuum at 50° C. overnight to obtain 10.33 g of Pleuromutilin mesylate as a white precipitate (86% yield).

### Example 4

### Preparation of Pleuromutilin Mesylate

A 250 ml round bottom flask was charged with Pleuromutilin (10 g, 0.0263 mol), Et₃N (4.2 ml, 0.02895 mol) and methyl isobutyl ketone (MIBK) (10 vol, 100 ml). Methane sulfonyl chloride (1.1 eq, 2.25 ml, 0.029 mol) was added at room temperature. The solution became a massive white slurry. The reaction mixture was stirred for 1.5 hours at room temperature, and then filtered and washed with MIBK (10 ml×2). The filtrate was evaporated to dryness. The residue was crystallized in isopropyl alcohol (IPA) (40 ml, 4 vol) at room temperature overnight. The obtained precipitate was filtered under reduced pressure and dried under vacuum at 50° C. overnight to obtain 9.33 g of Pleuromutilin mesylate as a white solid (77% yield, 99.3% purity by assay).

### Example 5

### Preparation of Pleuromutilin Mesylate

A 250 ml round bottom flask was charged with Pleuromutilin (10 g, 0.0263 mol), Et₃N (4.2 ml, 0.02895 mol) and Toluene (10 vol, 100 ml). Methane sulfonyl chloride (1 eq, 2 ml, 0.02575 mol) was added at room temperature. The turbid solution became a massive white slurry. The reaction mixture was stirred for 2 hours at room temperature. Water (100 ml) was added to the reaction mixture and the 2 phases were separated. The organic phase was evaporated until dryness and the residue was crystallized in isopropyl alcohol (IPA) (50 ml, 5 vol.) at room temperature over night. The obtained precipitate was filtered under reduced pressure and dried under vacuum at 50° C. overnight to obtain 9.72 g of Pleuromutilin mesylate as a white solid (80% yield, 100.5% purity by assay).

### Example 6

### Preparation of Pleuromutilin Mesylate

A 250 ml round bottom flask was charged with Pleuromutilin (10 g, 0.0263 mol), Et₃N (4.2 ml, 0.02895 mol) and Ethyl acetate (15 vol, 150 ml). Methane sulfonyl chloride (1.1 eq, 2.25 ml, 0.03 mol) was added at room temperature. The solution became a massive white slurry. The reaction mixture was stirred overnight at room temperature, and then filtered and washed with Ethyl acetate (10 ml). The Ethyl acetate was evaporated until dryness.

Isopropyl alcohol (IPA) (5 vol) was added to the residue and the mixture was stirred at room temperature for 5 hours. The obtained precipitate was filtered under reduced pressure, washed with IPA (5 ml×2) and dried under vacuum at 50° C. overnight to obtain 5.51 g of Pleuromutilin mesylate as a white precipitate (41.7% yield).

### Example 7

### Preparation of Pleuromutilin Mesylate

A 250 ml round bottom flask was charged with Pleuromutilin (10 g, 0.0263 mol), Et₃N (4.2 ml, 0.02895 mol) and Isobutyl acetate (15 vol, 150 ml (Fluka)). Methane sulfonyl chloride (1.1 eq, 2.25 ml, 0.03 mol) was added at room temperature. The solution became a massive white slurry. The reaction mixture was stirred for 1.5 hours at room temperature and then filtered and washed with Isobutyl acetate (10 ml). The Isobutyl acetate was evaporated until dryness to give 15.12 g oil.

Cyclohexane (10 vol) was added to the oil and the mixture was stirred at room temperature overnight. The obtained precipitate was filtered under reduced pressure, washed with cyclohexane (50 ml×2) and dried under vacuum at 50° C. overnight to obtain 10.33 g of Pleuromutilin mesylate as a white precipitate (78.5% yield).

### Example 8

### Preparation of Pleuromutilin Mesylate

A 250 ml round bottom flask was charged with Pleuromutilin (10 g, 0.0263 mol), Et₃N (7.6 ml, 0.05263 mol) and Ethyl acetate (15 vol, 150 ml). Methane sulfonyl chloride (1.5 eq, 3 ml, 0.0386 mole) was added at room temperature. The solution became a massive white slurry. The reaction mixture was stirred for 1.5 hours at room temperature and then filtered and washed with Ethyl acetate (20 ml×2). The Ethyl acetate was evaporated until dryness to give 16 g oil.

Isopropyl alcohol (IPA) (5 vol) was added to the residue and the mixture was stirred at room temperature overnight. The obtained precipitate was filtered under reduced pressure, washed with IPA (10 ml×2) and dried under vacuum at 50° C. overnight to obtain 4.38 g of Pleuromutilin mesylate as a white precipitate (35% yield).

### Example 9

### Preparation of Pleuromutilin Mesylate

A 250 ml round bottom flask was charged with Pleuromutilin (10 g, 0.0263 mol), Et₃N (1.5 eq, 5.7 ml, 0.04 mol) and acetone (7.5 vol, 75 ml). Methane sulfonyl chloride (1.5 eq, 3 ml, 0.04 mol) was added at room temperature. The solution became a massive white slurry. The reaction mixture was stirred for 5 hours at room temperature, filtered, and washed with acetone (5 ml). A conversion of 76% was obtained.

### Example 10

### Preparation of Pleuromutilin Mesylate

A 1 L round bottom flask was charged with Pleuromutilin (30 g, 0.079 mol), Et₃N (2 eq, 22.9 ml, 0.158 mol) and tetrahydrofuran (THF) (10 vol, 300 ml). Methane sulfonyl chloride (2 eq, 12.2 ml, 0.158 mol) was added at room temperature. The solution became a massive white slurry. The reaction mixture was stirred overnight at room temperature, filtered, and washed with THF (20 ml×2). A conversion of 94% was obtained.

### Example 11

### Preparation of Tropine Mesylate

To a 500 ml round bottom flask with a mechanical stirrer and nitrogen inlet charge, Tropine (10 g, 70.81 mmol), methyl isobutyl ketone (MIBK) (200 ml) and triethylamine (14.31 g, 141.63 mmol) were charged. A clear solution was obtained. The reaction was cooled to −10° C. (in an ice water/acetone bath). Methane sulfonyl chloride (18.49 g, 164.55 mmol) was added dropwise while the reaction was being cooled to −10° C. Solid started to precipitate. The reaction was allowed to reach room temperature and the reaction was stirred at room temperature overnight. The reaction was monitored by HPLC and full conversion was obtained (the product was not isolated).

### Example 12

### Preparation of Tropine Mesylate

To a 250 ml round bottom flask with a mechanical stirrer, Tropine (10 g, 70.81 mmol), Ethyl acetate (100 ml) and triethylamine (10 g, 99 mmol) were charged. A diluted slurry was obtained. The reaction was cooled to −3° C. (in an ice water/acetone bath). Methane sulfonyl chloride (10 g, 87 mmol) dissolved in Ethyl acetate (10 ml) was added dropwise while the reaction was being cooled to −3° C. Solid started to precipitate massively. The reaction was allowed to reach room temperature and the reaction was stirred at room temperature overnight. The reaction was monitored by HPLC and 87% conversion was obtained (the product was not isolated; 63% yield by assay).

### Example 13

### Preparation of Tropine Mesylate

To a 250 ml round bottom flask with a mechanical stirrer, Tropine (10 g, 70.81 mmol), isobutyl acetate (100 ml) and triethylamine (10 g, 99 mmol) were charged. A diluted slurry was obtained. The reaction was cooled to −3° C. (in an ice water/acetone bath). Methane sulfonyl chloride (10 g, 87 mmol) dissolved in Isobutyl acetate (10 ml) was added dropwise while the reaction was cooled to −3° C. Solid started to precipitate massively. The reaction was allowed to reach room temperature and the reaction was stirred at room temperature overnight. The reaction was monitored by HPLC and 83% conversion was obtained (the product was not isolated; 66% yield by assay).

### Example 14

### Preparation of Tropine Mesylate

To a 150 ml reactor, Tropine (10 g, 70.81 mmol), acetone (100 ml) and triethylamine (15 ml, 212 mmol) were charged. The reaction mixture was stirred at room temperature for 20 min, cooled to −5° C. and methane sulfonyl chloride (11.6 g, 101 mmol) was added dropwise via a syringe pump. The temperature was raised to 5° C. and the reaction was stirred at 5° C. overnight. The reaction was monitored by HPLC; the product was not isolated (79% yield by assay).

### Example 15

### Preparation of Tropine Mesylate

To a 150 ml reactor, tropine (10 g, 70.81 mmol), tetrahydrofuran (THF) (100 ml) and triethylamine (15 ml, 212 mmol) were charged. The reaction was stirred at room temperature for 30 minutes, cooled to −5° C. and methane sulfonyl chloride (11.6 g, 101 mmol) was added dropwise via a syringe pump. The temperature was raised to 15° C., and the reaction was stirred at 15° C. overnight. The reaction was monitored by HPLC; the product was not isolated (88% yield by assay).

### Example 16

### Preparation of Tropine Mesylate

To a 250 ml flask with a mechanical stirrer, tropine (10 g, 70.81 mmol), methyl isobutyl ketone (MIBK) (100 ml) and triethylamine (15 ml, 212 mmol) were charged. The reaction was stirred and cooled in an ice/acetone bath and methane sulfonyl chloride (11.6 g, 101 mmol) was added dropwise via syringe pump. The temperature was raised to room temperature and the reaction was stirred at room temperature overnight. The reaction was monitored by HPLC; the product was not isolated (84% yield by assay).

### Example 17

### Preparation of Retapamulin

A 3 neck-flask (100 ml) was charged with tropine thiol (5-10 mmol), a solvent (5 vol) and a base (2.5 eq). The solvents and bases are listed in Table 1 below. Pleuromutilin mesylate (11 mmol) was then added to the flask in portions. The resulting combination was stirred at room temperature to 40° C. for 2-24 hours until full conversion to Retapamulin was achieved.

TABLE 1
Solvents and Bases Used in the Procedure
for preparing Retapamulin

| Trial | Solvent | Base |
|---|---|---|
| 1 | Acetone | K₂CO₃ |
| 2 | EtOAc | K₂CO₃ |
| 3 | MIBK | K₂CO₃ |
| 4 | 2-BuOH | K₂CO₃ |
| 5 | MTBE | K₂CO₃ |
| 6 | Toluene | K₂CO₃ |
| 7 | IPA | K₂CO₃ |
| 8 | MIBK | Na₂CO₃ |
| 9 | toluene | KOH |
| 10 | MIBK | KOH |
| 11 | Acetone | KOH |
| 12 | EtOAc | KOH |
| 13 | IPA | KOH |
| 14 | 2-BuOH | KOH |
| 15 | MTBE | KOH |
| 16 | MIBK | KOH |
| 17 | MIBK | Et₃N |
| 18 | MIBK | t-BuONa |
| 19 | MIBK | MeONa |

### Example 18

### Preparation of Retapamulin

In a 500 ml round bottom flask, tropine thiol (15 g), methyl isobutyl ketone (8 vol) and triethylamine (2.4 eq.) were charged. The mixture was stirred at 40° C. for 15 minutes. Pleuromutilin mesylate (1 eq.) was added. The mixture was stirred as a slurry at 40° C. for 12 hours. Water (7 vol) was added and the pH was adjusted to 8.5 using 4N HCl. The phases were separated. Water (7 vol) was added to the organic phase, the pH was adjusted to 8.2, and the phases separated again. The organic phase was extracted with water (7 vol) and the pH was adjusted to 1.5. After separation, the pH of the aqueous phase was adjusted to 12.5 with 4N NaOH. The mixture was stirred at room temperature for 20 hours. The product was vacuum filtered and washed with water. The collected crystals were dried in a 55° C. vacuum oven to yield 80% Retapamulin at 99.8% purity as determined by HPLC.

What is claimed is:

1. A process for preparing a tropine derivative of the following Formula B: 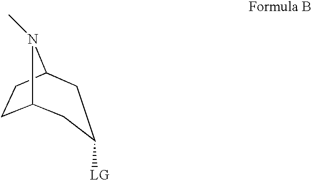
comprising: replacing the hydroxyl group of tropine with a leaving group (LG) in the presence of an organic solvent selected from the group consisting of C₂-C₆ ketones, C₂-C₇ esters and C₄-C₈ ethers.

2. The process according to claim 1, wherein the hydroxyl group of the tropine is replaced with a leaving group by a reagent selected from the group consisting of methane sulphonyl chloride, p-toluenesulfonyl chloride, thionyl chloride, thionyl bromide, 4-nitrobenzenesulfonyl chloride, benzenesulfonyl chloride, acetyl chloride, and acetic anhydride.

3. The process according to claim 1, wherein the tropine derivative is tropine mesylate.

4. The process according to claim 2, wherein the hydroxyl group of tropine is replaced with a leaving group (LG) in the presence of an organic solvent and an organic base.

5. The process according to claim 4, wherein the organic base is triethyl amine or tributyl amine.

6. The process according to claim 1, wherein the organic solvent is selected from the group consisting of acetone, methyl isobutyl ketone, ethyl acetate, isobutyl acetate and tetrahydrofuran.

7. The process according to claim 6, wherein the organic solvent is tetrahydrofuran or methyl isobutyl ketone.

8. A process for preparing Retapamulin comprising: obtaining a tropine derivative according to claim 1 and further converting the tropine derivative to Retapamulin.

9. The process according to claim 8, wherein the step of converting the tropine derivative to Retapamulin comprises: combining the tropine derivative with an S-donor to obtain tropine thiol and further condensing the tropine thiol with a pleuromutilin derivative to obtain Retapamulin.

10. A process for preparing Retapamulin, comprising: combining a base, tropine thiol in a free base or salt form, an organic solvent, and a pleuromutilin derivative of Formula A:

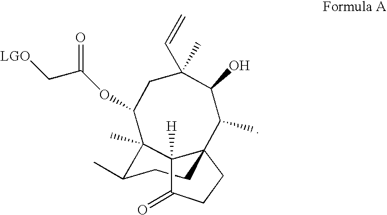

11. The process according to claim 10, wherein the organic solvent is selected from the group consisting of butanol, isopropanol, methyl tert butyl ether, acetone, methyl isobutyl ketone, acetonitrile, ethyl acetate and toluene.

12. The process according to claim 10, wherein the base is an alkali base, a carbonate, or a trialkyl amine.

13. The process according to claim 12, wherein the base is selected from the group consisting of t-BuONa, KOH, K₂CO₃, Na₂CO₃, and Et₃N.

14. The process according to claim 10, wherein the pleuromutilin derivative is obtained by a process comprising: replacing the hydroxyl group in the β position relative to the carbonyl group of pleuromutilin with a leaving group (LG) in the presence of an organic solvent selected from the group consisting of C₆-C₉ aromatic hydrocarbons, C₂-C₆ ketones, C₂-C₇ esters and C₄-C₈ ethers.

15. The process according to claim 14, wherein the hydroxyl group of the pleuromutilin derivative is replaced with the leaving group by a reagent selected from the group consisting of methane sulphonyl chloride, p-toluenesulfonyl chloride, thionyl chloride, thionyl bromide, 4-nitrobenzenesulfonyl chloride, benzenesulfonyl chloride, acetyl chloride, and acetic anhydride.

16. The process according to claim 10, wherein the pleuromutilin derivative is pleuromutilin mesylate.

17. The process according to claim 14, wherein the hydroxyl group in the β position relative to the carbonyl group of pleuromutilin is replaced with a leaving group (LG) in the presence of an organic solvent and an organic base.

18. The process according to claim 17, wherein the organic base is triethyl amine or tributyl amine.

19. The process according to claim 14, wherein the hydroxyl group in the β position relative to the carbonyl group of pleuromutilin is replaced with a leaving group (LG) in the presence of an organic solvent selected from the group consisting of toluene, ethyl acetate, isobutyl acetate, methyl isobutyl ketone, acetone and tetrahydrofuran.

20. The process according to claim 19, wherein the hydroxyl group in the β position relative to the carbonyl group of pleuromutilin is replaced with a leaving group (LG) in the presence of acetone.

21. The process according to claim 10, wherein the Retapamulin obtained is substantially pure.
